(19) United States
(12) Patent Application Publication
(10) Pub. No.: US 2005/0131045 A1
(43) Pub. Date: Jun. 16, 2005

(54) Novel crystal forms of ondansetron, processes for their preparation, pharmaceutical, compositions containing the novel forms and methods for treating nausea using them

(76) Inventors: Judith Aronhime, Rehovot (IL); Sandor Molnar, Debrecen (HU); Csaba Szabo, Debrecen (HU); Erzsebet Meszaros Sos, Debrecen (HU); Szabolcs Salyi, Debrecen (HU); Tivadar Tamas, Debrecen (HU)

Correspondence Address: KENYON & KENYON, 1500 K STREET, N.W., SUITE 700, WASHINGTON, DC, 20005, US

(21) Appl. No.: 10/952,165

(22) Filed: Sep. 29, 2004

Related U.S. Application Data
(63) Continuation-in-part of application No. 10/425,210, filed on Apr. 29, 2003.
(60) Provisional application No. 60/376,395, filed on Apr. 30, 2002.

(51) Int. Cl. A61K 31/4178; C07D 43/02

(52) U.S. Cl. 514/397; 548/311.4

(57) ABSTRACT

Ondansetron crystalline Forms A, B, and B1 are useful in the treatment of nausea and vomiting. Some of the novel forms have uniquely high melting points. The novel forms are stable against thermally induced polymorphic transition from 30° C. up to their melting points.

### CROSS REFERENCE TO RELATED APPLICATION

This application is a continuation-in-part of U.S. Ser. No. 10/425,210 filed Apr. 29, 2003 which claims the benefit under 35 U.S.C. § 1.119(e) of Provisional Application Ser. No. 60/376,395, filed Apr. 30, 2002, and both are incorporated herein by reference.

### FIELD OF THE INVENTION

The present invention relates to (±) 1,2,3,9-tetrahydro-9-methyl-3-[2-methyl-1h-imidazol-1-yl)methyl]-4h-carbazol-4-one (ondansetron). More particularly, it relates to newly discovered high melting crystalline forms of ondansetron, to another newly discovered crystalline form, to processes for producing the new forms, to pharmaceutical compositions containing them and methods of treating nausea and vomiting using them.

### BACKGROUND OF THE INVENTION

(±) 1,2,3,9-Tetrahydro-9-methyl-3-[2-methyl-1H-imidazol-1-yl)methyl]-4H-carbazol-4-one having the molecular structure 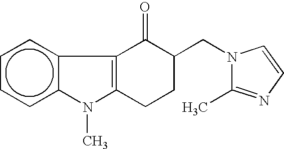
and formula C₁₈H₁₉N₃O is a selective 5-HT₃ receptor antagonist. It is a nitrogen-containing compound capable of existence in free base and salt forms. The free base is known by the generic name ondansetron. Ondansetron is useful for reducing nausea in patients undergoing chemotherapy. Grunberg, S. M.; Hesketh, P. J. “Control of Chemotherapy-Induced Emesis” N. Engl. J. Med. 1993, 329, 1790-96. It is approved by the United States Food and Drug Administration for prophylactic treatment of nausea and vomiting associated with some cancer chemotherapy, radiotherapy and postoperative nausea and/or vomiting. Ondansetron is commercially available in orally disintegrating tablets under the trade name Zofran® ODT.

The present invention relates to the solid state physical properties of ondansetron. According to the Merck Index 6977 (12th ed., Merck & Co: Whitehouse Station, N.J. 1996), ondansetron has a melting point (m.p.) range of 231-232° C.

U.S. Pat. No. 4,695,578 discloses several preparations of ondansetron. Commonly-assigned, co-pending U.S. patent application Ser. No. ______ [atty. ref. No. 2664/55602] also discloses a process for preparing ondansetron. The '578 patent and the [2664/55602] application are incorporated by reference in their entirety and, in particular, for their teachings how to synthesize ondansetron from commercially available and readily accessible starting materials.

In Example 4 of the '578 patent, 1,2,3,9-tetrahydro-9-methyl-3-[2-methyl-1H-imidazol-1-yl)methyl]-4H-carbazol-4-one was methylated at the 9-N position of the carbazol-4-one ring system with dimethylsulfate in N,N-dimethylformamide. Ondansetron forms as a solid in the reaction mixture. The isolated solid decomposes at 223-224° C.

In Example 7 of the '578 patent, ondansetron was made by displacing dimethylamine from 3-[(dimethylamino)methyl]-1,2,3,9-tetrahydro-9-methyl-4H-carbazol-4-one with 2-methylimidazole in water (although the mechanism of the reaction is not necessarily a simple substitution). The precipitated crude product with a melting point of 221-221.5° C. was recrystallized from methanol to give ondansetron with a melting point of 231-232° C.

In Example 8 of the '578 patent, ondansetron was prepared by Michael-type addition of 2-methylimidazole to 1,2,3,9-tetrahydro-9-methyl-3-methylene-4H-carbazol-4-one. The product was recrystallized from methanol to give ondansetron that had a melting point of 232-234° C.

In Example 18(ii) of the '578 patent, ondansetron with a melting point of 228-229° C. was prepared by substitution of 2-methylimidazole for chloride in 3-(chloromethyl)-1,2,3,9-tetrahydro-9-methyl-4H-carbazol-4-one followed by column chromatography.

In Example 19 of the '578 patent, ondansetron with a melting point of 227-228.5° C. was prepared by DDQ oxidation of 2,3,4,9-tetrahydro-9-methyl-3-[(2-methyl-1H-imidazol-1-yl)methyl]-1H-carbazole maleate followed by column chromatography.

In Example 20 of the '578 patent, ondansetron with a melting point of 232-234° C. was prepared by DDQ oxidation of 2,3,4,9-tetrahydro-9-methyl-3-[(2-methyl-1H-imidazol-1-yl)methyl]-1H-carbazol-4-ol, followed by column chromatography.

In U.S. Pat. Nos. 4,983,621, 4,783,478 and 4,835,173, ondansetron was prepared as described in Example 7 of the '578 patent to produce crude and recrystallized ondansetron with identical melting point ranges.

In U.S. Pat. No. 4,957,609, ondansetron was prepared by intramolecular palladium catalyzed coupling of 3-[2-iodophenyl)methylamino]-6-[(2-methyl-1H-imidazol-1-yl)methyl]-2-cyclohexen-1-one followed by column chromatography. The product decomposed at 215-216° C.

In U.S. Pat. No. 4,739,072, ondansetron was prepared by a reaction involving zinc catalyzed cyclization of 6-[(2-methyl-1H-imidazol-1-yl)methyl]-3-(2-methyl-2-phenylhydrazino)-2-cyclohexen-1-one. Column chromatography yielded a product that melted at 216-218° C. Recrystallization of the chromatographed product from methanol gave ondansetron that melted in the range 227.5-228.5° C.

As the foregoing summary of some known processes for preparing ondansetron makes evident, the reported melting points of ondansetron vary widely, from 215° C. with decomposition to as high as 234° C. without decomposition, depending on how the ondansetron was prepared and isolated. It appears that ondansetron that has been crystallized from methanol in the past melted in a more narrow and consistent temperature range according to these reports (m.p. 227-234° C.) than chromatographed material which appears to have melting points scattered over a wide range (215-234° C.).

We have now discovered and characterized novel high melting crystalline forms of ondansetron and another crystalline form that melts in a temperature more typical of ondansetron that has been produced by prior methods.

There is a need for new crystalline forms of ondansetron. The discovery of new crystalline forms of a pharmaceutical compound provides an opportunity to improve the performance characteristics of a pharmaceutical product. It enlarges the repertoire of materials that a formulation scientist has available for designing, for example, a pharmaceutical dosage form of a drug with a targeted release profile or other desirable characteristic.

### SUMMARY OF THE INVENTION

One aspect of the present invention is directed to crystalline Form B of ondansetron. Ondansetron Form B has a uniquely high melting point of 244±2° C. and is stable toward thermally induced polymorphic transition between 30° C. and 180° C. Form B is identifiable by powder X-ray crystallography as well as its thermal properties. Form B can be prepared under controlled conditions by precipitation from certain alcohol solvents.

Another aspect of the present invention is directed to crystalline Form B1 of ondansetron. Ondansetron Form B1 has a melting point of about 235° C. to about 240° C. Form B1 is characterized by X-ray powder diffraction peaks at 7.3, 10.9, 20.3, and 20.8±0.2 degrees two-theta.

Yet another aspect of the present invention is directed to crystalline Form A of ondansetron which is readily identifiable by its powder X-ray diffraction pattern. Ondansetron Form A also is stable toward thermally induced polymorphic transition between 30° C. and 180° C. Form A can be prepared under controlled conditions by precipitation from select organic solvents and mixtures of those organic solvents and water.

The present invention further provides pharmaceutical compositions comprising ondansetron Form A, ondansetron Form B, ondansetron Form B1 and mixtures thereof.

Yet further, the present invention provides methods for treating and/or preventing nausea and vomiting with ondansetron Form A, ondansetron Form B, and ondansetron Form B1. In particular ondansetron Forms A, B, and B1 are useful for treating and/or preventing nausea and vomiting associated with surgery, emetogenic cancer chemotherapy and radiotherapy.

### BRIEF DESCRIPTION OF THE DRAWINGS

FIG. 1 is a differential scanning calorimetry thermogram of ondansetron Form B.

FIG. 2 is a characteristic powder X-ray diffraction pattern of ondansetron Form B.

FIG. 3 is a differential scanning calorimetry thermogram of ondansetron Form A.

FIG. 4 is a characteristic powder X-ray diffraction pattern of ondansetron Form A.

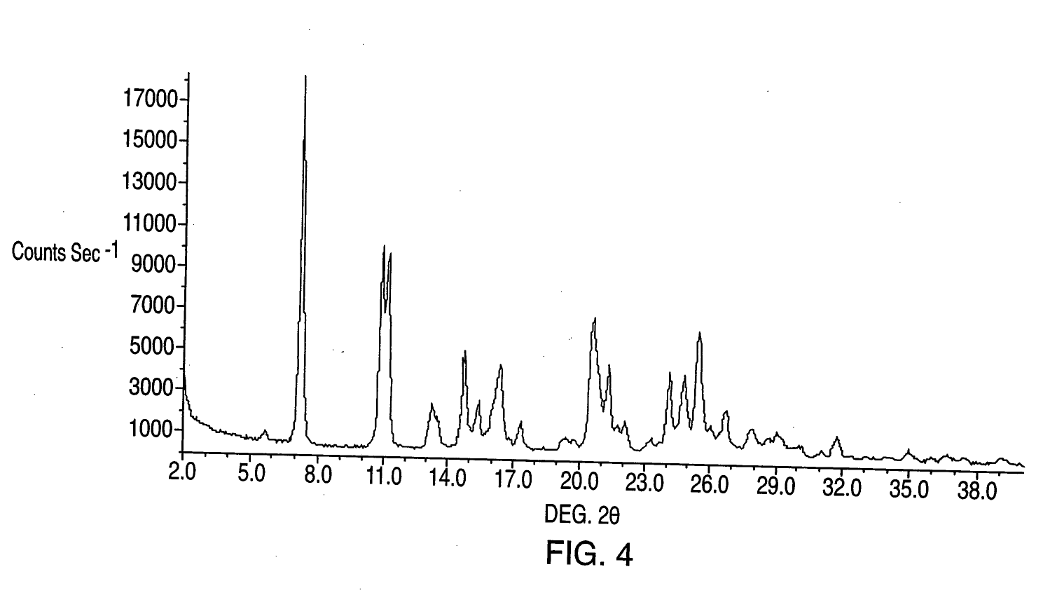

FIG. 5 is a differential scanning calorimetry thermogram of ondansetron Form B1.

FIG. 6 is a characteristic powder X-ray diffraction pattern of ondansetron Form B1.

### DETAILED DESCRIPTION OF THE INVENTION

In one aspect, the present invention provides a new thermally stable crystalline form of ondansetron, designated Form B. Form B has been characterized by powder X-ray diffraction (“PXRD”) analysis, and thermal methods including differential scanning calorimetry (“DSC”) and thermogravimetric analysis (“TGA”). PXRD patterns and differential thermograms are provided as figures. Where relevant, TGA results are discussed in the written portion of the disclosure.

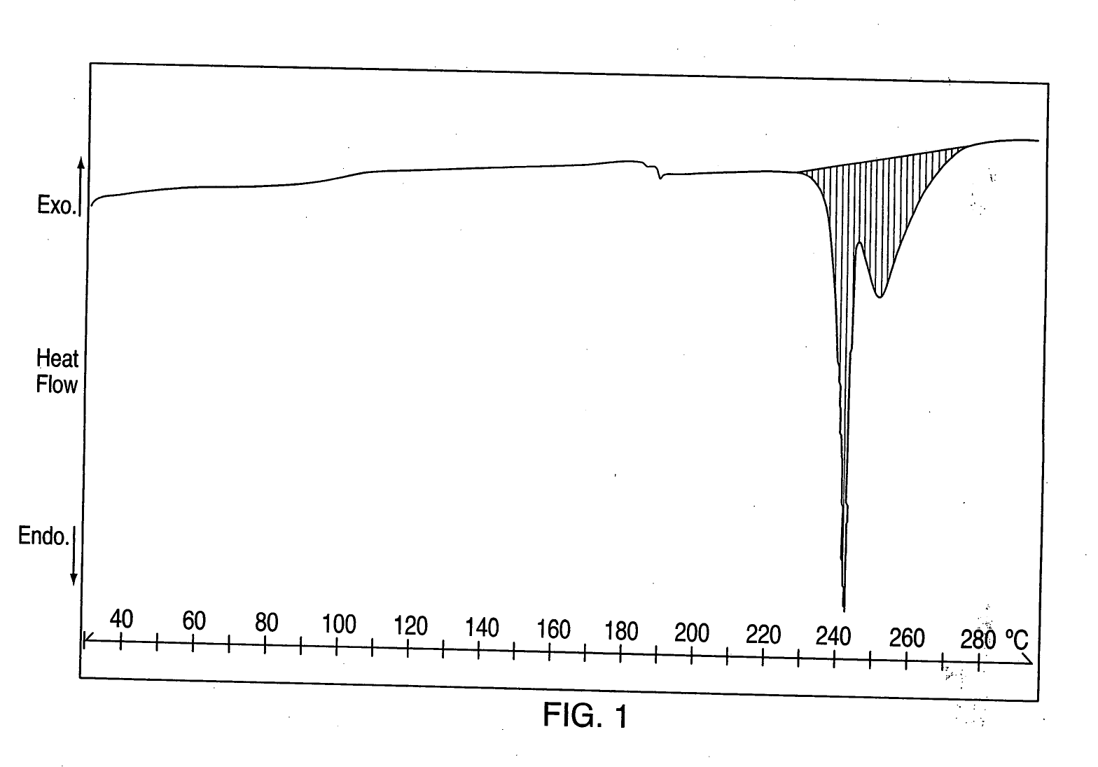

Referring to FIG. 1, the differential thermogram of ondansetron Form B demonstrates the unique thermal stability of this crystalline form. FIG. 1 possesses a sharp melting endotherm with a maximum at 244° C. Variation in the temperature of the maximum endotherm of melting obtained from like samples of Form B analyzed on different commercial calorimeters using the same heating rate should be considerably less than ±2° C. However, capillary melting points typically are not measured or recorded with accurately determined heating rates. Different heating rates combined with thermal inertia can cause the capillary melting point to deviate from the true melting point of a sample. Thus, it is considered that ondansetron that produces a thermal analysis result, e.g. measured melting point, maximum melting endotherm, inflection point in heat absorption curve and the like, that is indicative of melting at 244±2° C. is consistent with its identity as Form B. The magnitude of the melting endotherm was estimated to be 140.11 J g⁻¹ but overlap with another endotherm prevented accurate determination of the heat of fusion.

Above the melting endotherm and partially overlapping it, there is a broad endotherm caused by volatilization or chemical decomposition of ondansetron. At temperatures below the melting endotherm, the differential thermogram is flat. This characteristic is consistent with an absence of a polymorphic transition before melting. Therefore, Form B appears to be stable toward thermally induced polymorphic transitions from 30° C. to 180° C., although transitions that are neither detectably endothermic or endothermic could occur. The thermal analysis was conducted under a dry, inert atmosphere. Therefore, the susceptibility of Form B to solvent induced transitions, including vapor induced transitions in this temperature range, also is not precluded.

Differential scanning calorimetry was performed using a Mettler Toledo 821 STAR^{e} system. Samples of 3-5 mg were analyzed in aluminum crucibles with lids loosely fitted. Scans were performed from 30 to 300° C. at a ramp rate of 10° C. min⁻¹ under a nitrogen purge with a 40.0 ml min⁻¹ flow rate. The sample that produced the thermogram reproduced in FIG. 1 weighed 5.05 mg.

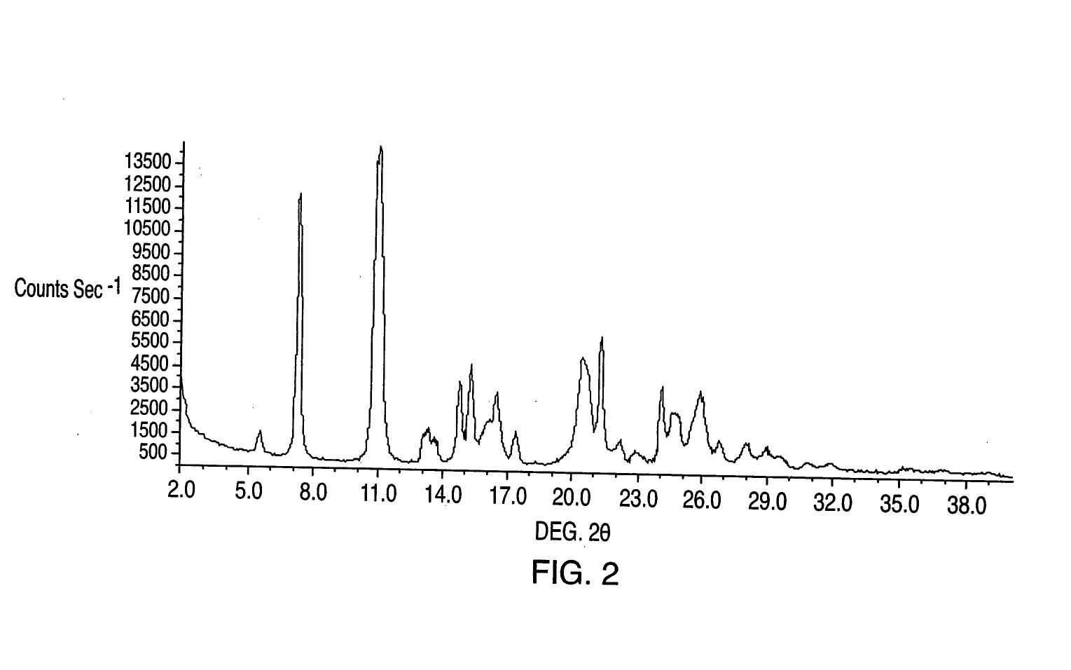

The PXRD pattern (FIG. 2) of ondansetron Form B is unique. Form B may be characterized by the PXRD characteristics set forth in Table 1 which distinguish it from other forms.

TABLE 1
Peak Position (°2θ)^{a}

11.0
11.2
14.9
15.5
15.9
16.5
20.6
21.4
23.1
23.5
24.2
24.7
24.8
25.8
26.9
28.1
^{a}expected variation between instruments: ±1.0°.

PXRD patterns were produced on a Scintag X-ray powder diffractometer model X'TRA equipped with a copper anode tube and a solid state detector. Samples were prepared by gentle and thorough grinding in an agate mortar to reduce preferential orientation. No loss in crystallinity of samples prepared by grinding was noted. The powdered sample was poured into the round cavity of a sample holder and pressed with a glass plate to form a smooth surface. Continuous scans were run from 2 to 40°2θ at 3° min.⁻¹. Reported peak positions are considered accurate to within ±0.05°. Those skilled in the art of X-ray crystallography will appreciate that peak positions determined on different instruments may vary by as much as ±1°.

The loss on drying (“LOD”) of ondansetron Form B was found to be about 2%, which is less than the amount calculated for a hypothetical hemi-hydrate (or C₁-C₃ alcohol hemi-solvate) and is considered consistent with unsolvated ondansetron having adsorbed moisture. LOD was measured by TGA using A Mettler TG50: Sample weight: 7-15 mg, heating rate: 10° C. min.^{-1.} Standard alumina crucibles were used.

Ondansetron Form B has been prepared under controlled conditions. It is only possible to describe methods which have successfully yielded Form B. Other conditions by which ondansetron Form B is produced may be found by routine experimentation.

Ondansetron Form B may be prepared by crystallizing ondansetron from a solution in a C₁-C₃ alcohol, in particular, methanol, ethanol, propan-1-ol, propan-2-ol and mixtures thereof. Ondansetron is dissolved in the C₁-C₃ alcohol, preferably in an amount sufficient to produce from about a 50 mM to about a 300 mM solution, more preferably from about an 85 mM to about a 150 mM solution. Ondansetron has limited solubility in these alcohols at room temperature. Consequently, it may be necessary to heat the mixture in order to fully dissolve it. Preferably, the mixture is refluxed until the mixture becomes a clear solution. The solution is preferably free of solid ondansetron that could potentially seed the mixture causing precipitation of ondansetron in a crystalline form other than Form B or co-crystallization of Form B with another form. Preferably, the Form B obtained by crystallization from the alcohol solution contains less than or equal to about 5% other crystalline forms of ondansetron, more preferably Form B contains less than or equal to about 1% other crystalline forms of ondansetron.

Crystallization of Form B from the solution can occur spontaneously on standing at room temperature. If the mixture has been heated, cooling of the solution can cause supersaturation that induces crystallization of Form B. Crystallization also can be induced by seeding with a crystal of ondansetron Form B. Maximum recovery of ondansetron Form B is achieved by cooling the mixture to below ambient temperature, such as from about 20° C. to about 0° C. Another means of enhancing the yield of Form B is to evaporate some of the alcohol after the starting ondansetron has completely dissolved. An example showing the use of a combination of techniques for optimal recovery of Form B is provided below. It will be noted that the preferred solution concentrations are dilute. This is a consequence of the poor solubility of ondansetron in the lower alcohols from which Form B has been obtained. Cooling and/or partial evaporation of solvent is recommended to maximize recovery of the traces of dissolved ondansetron in solution after partial crystallization, though their use is not critical to practice of this invention.

After crystallization has been deemed sufficiently complete, the crystals are separated from the alcohol by conventional means such as filtration, decantation, centrifugation and the like. The crystals may be washed with solvent, such as cold methanol and dried under desiccating conditions such as 65° C. under aspirator or oil pump vacuum. Yields in the 70-90% range are typical, though they may be higher or lower.

Ondansetron Form B can be obtained in good polymorphic purity by following the preferred embodiments of the foregoing process. Preferably ondansetron Form B prepared by that process contains less than or equal to about 5% other crystalline forms of ondansetron, more preferably less than or equal to about 1% other crystalline forms of ondansetron. Less preferred process embodiments or other processes may yield ondansetron Form B in lesser degrees of purity, particularly if a seed of another polymorph is present. Mixtures containing as little as 25% ondansetron Form B, or less, may exhibit improved properties due to the presence of Form B and, therefore, such mixtures are considered to be improved by and to fall within the scope of the present invention. Of course, ondansetron Form B that is found in mixture with other substances, like pharmaceutical excipients, even as a minor component is specifically contemplated as a material embraced by ondansetron Form B that produces a thermal analysis result indicative of a melting point of 244±2° C.

In another aspect, the present invention provides a crystalline form of ondansetron, designated Form B1. Form B1 is characterized by X-ray powder diffraction peaks at 7.3, 10.9, 20.3, and 20.8±0.2 degrees two-theta. Also, Form B1 may be characterized by a DSC melting point of about 235° C. to about 240° C.

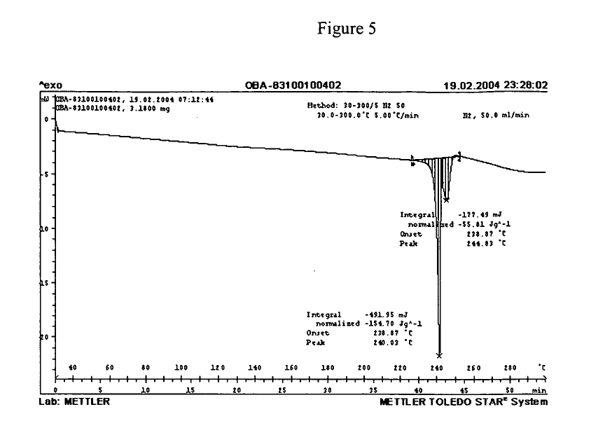

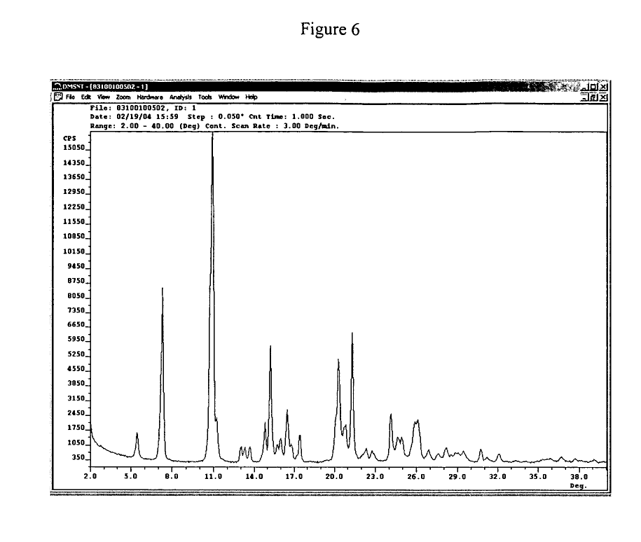

Form B1 may be further characterized by X-ray powder diffraction peaks at 14.8, 15.2, 15.7, 16.0, 24.1, 24.6, and 24.9±0.2 degrees two-theta. A typical X-ray powder diffraction pattern of Form B1 is shown in FIG. 6. A typical DSC thermogram of Form B1 is shown in FIG. 5.

Thermogravimetric analysis can also be applied to the characterization of Form B1. The loss on drying of Form B1 is typically about 2% by weight.

Form B1 has been prepared under controlled conditions. Ondansetron is dissolved in methanol, preferably in an amount sufficient to produce a solution of about 50 mM to about 300 mM concentration, more preferably about 85 mM to about 150 mM. Ondansetron has limited solubility in alcohol at room temperature. Consequently, it may be necessary to heat the mixture in order to fully dissolve it. Preferably, the mixture is refluxed until the mixture becomes a clear solution. The solution is preferably free of solid ondansetron that could potentially seed the mixture causing precipitation of ondansetron in a crystalline form other than Form B1 or co-crystallization of Form B1 with another form.

Crystallization of Form B1 from the solution can occur spontaneously on standing at room temperature. If the mixture has been heated, cooling of the solution can cause supersaturation that induces crystallization of Form B1. Crystallization also can be induced by seeding with a crystal of ondansetron Form B1. Maximum recovery of ondansetron Form B1 is achieved by cooling the mixture to below ambient temperature, such as about 20° C. to about 0° C., preferably about 5° C. to about 0° C. Another means of enhancing the yield of Form B1 is to evaporate some of the alcohol after the starting ondansetron has completely dissolved. An example showing the use of a combination of techniques for optimal recovery of Form B1 is provided below. It will be noted that the preferred solution concentrations are dilute. This is a consequence of the poor solubility of ondansetron in the lower alcohols from which Form B1 has been obtained. Cooling and/or partial evaporation of solvent is recommended to maximize recovery of the traces of dissolved ondansetron in solution after partial crystallization, though their use is not critical to practice of this invention.

After crystallization has been deemed sufficiently complete, the crystals are recovered from the alcohol by conventional means such as filtration, decantation, centrifugation and the like. The crystals may be washed with solvent, such as cold methanol and dried under desiccating conditions such as 65° C. under aspirator or oil pump vacuum. Yields in the 70-90% range are typical, though they may be higher or lower.

Ondansetron Form B1 can be obtained in good polymorphic purity by following the preferred embodiments of the foregoing process. Preferably ondansetron Form B1 prepared by that process contains less than or equal to about 5% by weight other crystalline forms of ondansetron, more preferably less than or equal to about 1% by weight other crystalline forms of ondansetron. Less preferred process embodiments or other processes may yield ondansetron Form B1 in lesser degrees of purity, particularly if a seed of another polymorph is present. Mixtures containing as little as 25% ondansetron Form B1, or less, may exhibit improved properties due to the presence of Form B1 and, therefore, such mixtures are considered to be improved by and to fall within the scope of the present invention. Of course, ondansetron Form B1 that is found in mixture with other substances, like pharmaceutical excipients, even as a minor component is specifically contemplated as a material embraced by ondansetron Form B1 that produces a thermal analysis result indicative of a melting point of about 235° C. to about 240° C.

In yet another aspect, the present invention provides ondansetron Form A. Form A has been characterized by PXRD, DSC and TGA using identical equipment and sample preparations as were used to characterize Form B.

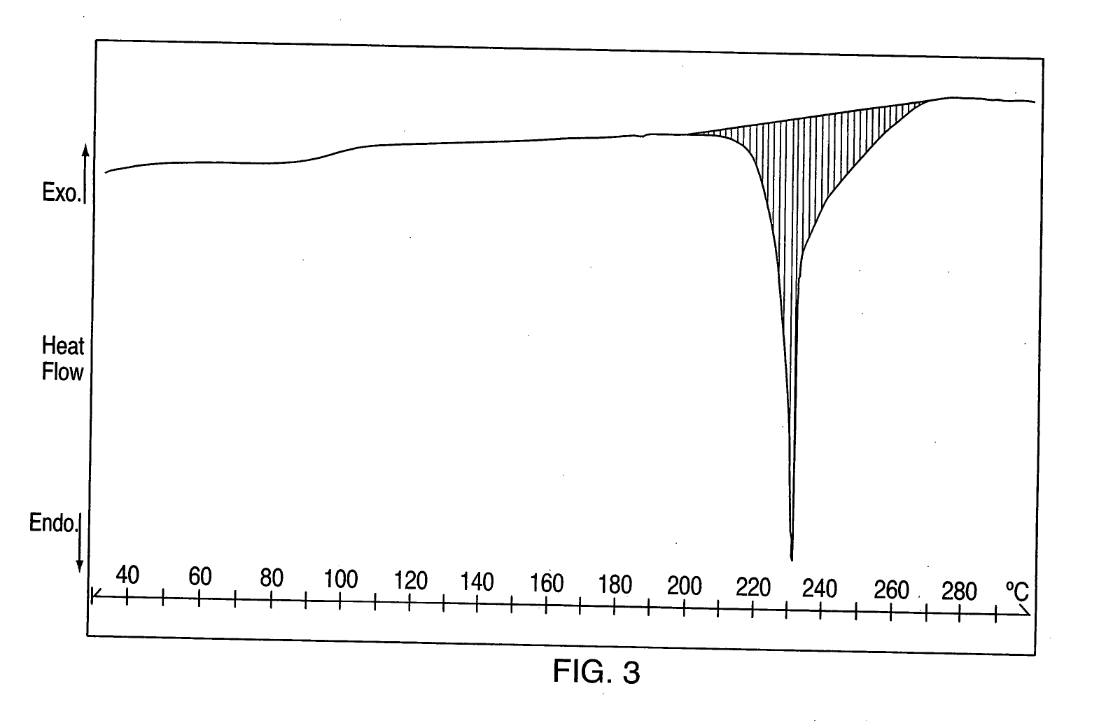

Referring to FIG. 3, the differential thermogram of Form A possesses a melting endotherm with a maximum at 230° C. At temperatures higher than 230° C., there is a broad endotherm overlapping the melting endotherm that is attributed to volatilization of the ondansetron. When Form A was heated in an “open pan” the broad overlapping endotherm was not observed. However, when Form B was heated in an open pan, its DSC thermogram was the same as the thermogram observed when Form B was heated in a closed pan. The DSC thermogram of Form A was made on the same equipment and using the same procedure (but for differences noted) as were used with Form B. The sample that produced the thermogram of FIG. 3 weighed 4.75 mg.

The PXRD pattern of ondansetron Form A also clearly distinguishes it from other forms. The positions of characteristic peaks in the PXRD pattern of Form A are set forth in Table 2.

TABLE 2
Peak Position (°2θ)^{a}

11.0
11.2
14.8
15.4
16.4
20.6
21.4
23.2
24.1
24.7
25.4
25.9
26.7
27.8
^{a}expected variation between instruments: ±1.0°.

Beginning with the PXRD characteristics common to Forms A, B, and B1, there are strong peaks at 7.0 and 11.0±1.0°2θ and other common peaks at 14.8, 24.2, and 24.7±1.0°2θ.

Significant differences between the forms are found in the 15-16° region and the 22-24° region of the patterns. In the 15-16° region, Form A has one peak, Form B has two peaks, and Form B1 has three peaks. In the 22-24° region, Form B1 has no peaks, Form A has one peak, and Form B has two peaks. Also, Form B1 is distinctive in that is has two strong peaks at 20.3°2θ and 20.8°2θ whereas forms A and B each have one peak at 20.6°2θ.

Like Form B, a sample of Form A was found to have an LOD of about 2%.

Form A has been prepared under controlled conditions. It is only possible to describe methods which have successfully yielded Form A. Other conditions by which ondansetron Form A is produced may be found by routine experimentation.

Form A may be prepared by crystallization from a wide variety of organic solvents and mixtures of organic solvents and water. Suitable organic solvents include C₄ and higher mono-, di- and polyhydroxylic alcohols; liquid aromatic compounds, such as benzene and toluene; acetic acid esters, such as ethyl acetate and butyl acetate; and polar aprotic solvents such as N,N-dimethylformamide (“DMF”). Preferred solvents are 1-butanol, ethyl acetate, butyl acetate, DMF and DMF-water mixtures. Especially preferred solvents are 1-butanol and DMF.

Ondansetron is preferably completely dissolved in the solvent before attempting to isolate Form A as a precipitate. The solubility of ondansetron in the solvent is a factor that effects the relative amounts of ondansetron and the solvent to be combined. Whereas the polarity of the solvents from which Form A can be crystallized is somewhat varied, the ratio of ondansetron to solvent varies significantly depending on solvent selection. When one of the especially preferred solvents is used, ondansetron is preferably added to the solvent in an amount sufficient to form a 50 mM to about 300 mM solution once it has completely dissolved.

Heating the mixture of ondansetron and the solvent is preferred to accelerate dissolution and increase solubility. More preferably, the mixture is heated to the reflux temperature of the solvent. Crystallization of Form A may occur spontaneously or it may be induced, for example by cooling, evaporation of solvent or seeding. A heated solution may be cooled to ambient temperature and a heated or ambient temperature solution may be cooled to low temperature, such as from 20° C. to 0° C.

After crystallization of Form A is deemed sufficiently complete, the crystals are separated from the solvent by conventional means such as filtration, decantation, centrifugation and the like. The crystals may be washed with an appropriate solvent and dried by conventional techniques.

Ondansetron Form A can be obtained in good polymorphic purity by following the preferred embodiments of the foregoing process. Preferably ondansetron Form A prepared by that process contains less than or equal to about 5% other crystalline forms of ondansetron, more preferably less than or equal to about 1% other crystalline forms of ondansetron. Less preferred process embodiments or other processes may yield ondansetron Form A in lesser degrees of purity, particularly if a seed of another polymorph is present. Mixtures containing as little as 25% ondansetron Form A, or less, may exhibit improved properties due to the presence of Form A and, therefore, such mixtures are considered to be improved by and to fall within the scope of the present invention. Of course, ondansetron Form A that is found in mixture with other substances, like pharmaceutical excipients, even as a minor component is specifically contemplated as a material embraced by ondansetron Form A.

Ondansetron Forms A, B and B1 have utility as the active agent in pharmaceutical compositions and dosage forms for prevention of nausea and vomiting associated with surgery, emetogenic cancer chemotherapy and radiotherapy. Ondansetron Forms A, B and B1 also are useful for preparing salts and solvates of ondansetron, such as the hydrochloride salt dihydrate that is currently administered to patients in the United States. To the extent that the atomic positions and molecular conformation of ondansetron do not significantly change with salt formation or solvation, such salts and solvates are considered to fall within the scope of the invention.

Ondansetron Forms A, B and B1 may be incorporated into pharmaceutical products for administration to a human or other mammal in need of suppression of vomiting. Pharmaceutical compositions and dosage forms may be formulated for transdermal delivery, enteral delivery or parenteral delivery. The most suitable route in any given case will depend on the nature and severity of the condition being treated and other circumstances that will be assessed by the caregiver. Pharmaceutical compositions for enteral delivery may be processed into tablets, powders, capsules, suppositories, sachets, troches and losenges as well as liquid solutions, suspensions, syrups and elixirs.

Exemplary of the many excipients known to pharmacy that can be included in enteral dosage forms, there are diluents, such as microcrystalline cellulose, lactose, starch, calcium carbonate, sugar, dextrose, dibasic calcium phosphate dihydrate, tribasic calcium phosphate, kaolin, maltodextrin and mannitol; binders such as acacia, alginic acid, carbomer, carboxymethylcellulose sodium, ethyl cellulose, gelatin, guar gum, hydroxyethyl cellulose, hydroxypropyl cellulose, hydroxypropyl methyl cellulose, maltodextrin, methylcellulose, polymethacrylates, povidone and sodium alginate; disintegrants such pregelatinized starch, alginic acid, carboxymethyl cellulose calcium, croscarmellose sodium, crospovidone and sodium starch glycolate; antioxidants and chelating agents such as alcohol, sodium benzoate, butylated hydroxy toluene, butylated hydroxyanisole and ethylenediamine tetraacetic acid; antimicrobial agents such as methylparaben and propylparaben, buffers such as guconic acid, lactic acid, citric acid or acetic acid, sodium guconate, sodium lactate, sodium citrate or sodium acetate and colorants such as titanium dioxide, iron oxide yellow or iron oxide red and sweeteners and flavorings such as sucrose, aspartame and strawberry flavor.

Pharmaceutical compositions containing ondansetron Forms A, B and B1 further include oral suspensions in which the ondansetron is dispersed in a liquid vehicle, optionally with viscosity modifiers, e.g. corn syrup; antimicrobial agents, e.g. sodium benzoate; buffering agents e.g. citric acid and sodium citrate; and flavoring agents e.g. strawberry flavoring.

Such pharmaceutical products further include injectable suspensions wherein the ondansetron is suspended in an aqueous or oily medium, optionally with an antimicrobial agent, and packaged in a single dose or multi-dose container.

An especially preferred pharmaceutical dosage form of ondansetron Form A, B and/or B1 is an orally disintegrating tablet. Orally disintegrating tablets can be formulated according to methods known in the art using pharmaceutical excipients that disperse or dissolve in saliva and do not retain the drug in solid form. Such excipients include gelatin and mannitol, and may further include antimicrobial agents such as methylparaben and propylparaben and sweetening agents and flavoring agents such as aspartame, and strawberry flavor.

Pharmaceutical compositions and dosage forms of this invention can be administered to a patient for the purpose of preventing nausea and vomiting associated with chemotherapy and postoperative nausea or vomiting in the manner that compositions containing known ondansetron have been administered. For this purpose, ondansetron Form A, B and/or B1 is administered preferably in an amount of from about 10 mg to about 50 mg per day, more preferably about 24 mg per day.

Having thus described the invention with respect to certain preferred embodiments, the invention will now be further illustrated with the following non-limiting examples.

### EXAMPLES

### Preparation of Ondansetron Form A

### Example 1

Ondansetron (2 g) was added to N,N-dimethylformamide (80 ml). The mixture was warmed to complete dissolution. The resulting clear solution was cooled to 20° C. and placed in a 2-8° C. refrigerator overnight. The next morning, the crystals were filtered off and dried at 60° C. in vacuum for one day to give ondansetron Form A (0.81 g, 41%).

### Example 2

Ondansetron (2 g) was added to 1-Butanol (30 ml). The mixture was warmed to reflux temperature. The resulting solution was cooled to 20° C. and then placed in a 2-8° C. refrigerator overnight. The next morning, the crystals were filtered off and dried at 60° C. under vacuum for one day to give ondansetron Form A (1.26 g, 63%).

### Preparation of Ondansetron Form B

### Example 3

Ondansetron (2 g) was added to ethanol (45 ml). The mixture was warmed to reflux temperature. The resulting clear solution was cooled to 20° C. and then placed in a 2-8° C. refrigerator overnight. The next morning, the crystals were filtered off and dried at 60° C. under vacuum for one day to give ondansetron Form B (1.76 g, 88%).

### Preparation of Ondansetron Form B1

### Example 4

Ondansetron (1.5 kg) was added to methanol (60 L). The mixture was warmed to reflux temperature. The clear hot solution was filtered through carbon (Norit-SX-1). Approximately a quarter of volume of methanol was distilled off. The solution was then cooled to 0-5° C. over 4 hours. The crystals were then filtered off, washed with methanol and dried at 65° C. under vacuum for one day to give ondansetron Form B1 (1.1 kg, 73%).

1. A crystalline form of ondansetron characterized by a thermal analysis result indicative of a melting point of about 235° C. to about 240° C.

2. A crystalline form of ondansetron characterized by a powder X-ray diffraction pattern having peaks at 7.3, 10.9, 20.3, 20.8±1.0 degrees two-theta.

3. The crystalline form of ondansetron of claim 2 further characterized by peaks in the X-ray diffraction pattern at 14.8, 15.2, 16.0, 24.1, 24.6, and 24.9±1.0 degrees two-theta.

4. The crystalline form of claim 3, characterized by a powder X-ray diffraction pattern substantially as depicted in FIG. 6.

5. The crystalline form of claim 1, characterized by a DSC thermogram substantially as depicted in FIG. 5.

6. The crystalline form of ondansetron of claim 1 or 2 containing less than or equal to about 5% by weight other crystalline forms of ondansetron.

7. The crystalline form of ondansetron of claim 6 containing less than or equal to about 1% by weight other crystalline forms of ondansetron.

8. A pharmaceutical composition or dosage form comprising the crystalline form of ondansetron of claim 1 or 2 and at least one pharmaceutical excipient.

9. The pharmaceutical composition or dosage form of claim 8 that is an orally disintegrating tablet.

10. A method of treating nausea and vomiting in a patient in need thereof comprising administering to the patient the pharmaceutical composition or dosage form of claim 8.

11. The crystalline form of ondansetron of claim 1 or 2 prepared by the process of:
a) dissolving ondansetron in methanol;
b) heating the ondansetron and methanol to form a solution having concentration of about 50 mM to about 300 mM;
c) crystallization ondansetron from the solution by cooling the methanol to about 0 degrees to about 5 degrees;
d) separating the ondansetron from the methanol; and
e) drying the ondansetron.
